(19) United States
(12) Patent Application Publication
(10) Pub. No.: US 2014/0342013 A1
(43) Pub. Date: Nov. 20, 2014

(54) CALCIUM PHOSPHATE CEMENT COMPOSITIONS THAT SET INTO HIGH STRENGTH POROUS STRUCTURES

(71) Applicant: Skeletal Kinetics, LLC, Cupertino, CA (US)

(72) Inventors: Jiawei He, Cupertino, CA (US); David C. Delaney, Capitola, CA (US)

(21) Appl. No.: 14/213,805

(22) Filed: Mar. 14, 2014

Related U.S. Application Data
(60) Provisional application No. 61/783,892, filed on Mar. 14, 2013.

(51) Int. Cl. A61L 24/02; A61L 24/00

(52) U.S. Cl. CPC A61L 24/02; A61L 24/0005; A61L 24/0036
USPC 424/549; 106/680; 424/602

(57) ABSTRACT

Calcium phosphate cement compositions are provided. Aspects of the cement compositions include a dry reactant component comprising a reactive α-tricalcium phosphate component, a multi-size pore forming calcium sulphate dihydrate component and a demineralized bone matrix component. During use, the dry reactant is combined with a setting fluid to produce a sellable composition that sets into a high strength porous product. Aspects of the invention further include the settable compositions themselves as well as kits for preparing the same. Methods and compositions as described herein find use in a variety of applications, including hard tissue repair applications.

### CROSS REFERENCE TO RELATED APPLICATIONS

Pursuant to 35 U.S.C. §119(e), this application claims priority to the filing date of U.S. Provisional Patent Application Ser. No. 61/783,892, filed on Mar. 14, 2013, the disclosure of which application is herein incorporated by reference in its entirety.

### INTRODUCTION

Calcium phosphate cements find use as structural materials in the orthopedic and dental fields. Such cements are typically prepared by combining a dry component(s) and a liquid to form a flowable paste-like material that is subsequently capable of setting into a solid calcium phosphate product. Materials that set into solid calcium phosphate mineral products are of particular interest as such products can closely resemble the mineral phase of natural bone and are susceptible to remodeling, making such products extremely attractive for use in orthopedics and related fields.

While a large number of different calcium phosphate cement formulations have been developed, there is a continued need for the development of yet more advanced formulations.

### SUMMARY

Calcium phosphate cement compositions are provided. Aspects of the cement compositions include a dry reactant component comprising a reactive α-tricalcium phosphate component, a multi-size pore forming calcium sulphate dihydrate component and a demineralized bone matrix component. During use, the dry reactant is combined with a setting fluid to produce a settable composition that sets into a high strength porous product. Aspects of the invention further include the settable compositions themselves as well as kits for preparing the same. Methods and compositions as described herein find use in a variety of applications, including hard tissue repair applications.

### DETAILED DESCRIPTION

Calcium phosphate cement compositions are provided. Aspects of the cement compositions include a dry reactant component comprising a reactive α-tricalcium phosphate component, a multi-size pore forming calcium sulphate dihydrate component and a demineralized bone matrix component. During use, the dry reactant is combined with a setting fluid to produce a sellable composition that sets into a high strength porous product. Aspects of the invention further include the settable compositions themselves as well as kits for preparing the same. Methods and compositions as described herein find use in a variety of applications, including hard tissue repair applications.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Calcium Phosphate Cements

As summarized above, the calcium phosphate cement compositions of the invention include the following components: a dry reactant that includes a reactive α-tricalcium phosphate component; a multi-size pore forming calcium sulfate dihydrate component and a demineralized bone matrix (DBM) component; and a liquid setting component.

### Dry Reactant Component

As summarized above, the dry reactant includes a reactive a-tricalcium phosphate component; a multi-size pore forming calcium sulfate dihydrate component and a demineralized bone matrix (DBM) component. Each of these components is now described in greater detail.

### Reactive α-Tricalcium Phosphate Component

The dry reactants include a reactive α-tricalcium phosphate (α-(Ca₃(PO₄)₂) component. In certain embodiments, the reactive α-tricalcium phosphate has a mean particle size (as determined using the Horiba LA-300 laser diffraction particle sizer (Version 3.30 software for Windows 95) (Irvine, Calif.)) of 8 μm or less and a narrow particle size distribution, e.g., as described in co-pending United States Published Patent Application 20070189951, the disclosure of which is herein incorporated by reference. As such, the α-tricalcium phosphate component of the cement has a mean particle size of 8 μm or less and a narrow particle size distribution. The mean particle size of this component may vary, ranging in some embodiments from 1 to 7 μm, such as from 1 to 6 μm, including from 1 to 5 μm, where the mean particle size in certain embodiments may be 1, 2, 3 and 4 μm, where in certain embodiments the mean particle size is 4 μm. By narrow particle size distribution is meant that the standard deviation of the particles that make up the particular reactant population (as determined using the Horiba LA-300 laser diffraction particle sizer (Version 3.30 software for Windows 95) (Irvine, Calif.)) is 4.0 or less, and in certain representative embodiments is 3.0 or less, e.g., 2.5 or less, including 2.0 μm or less. This particular reactant of the cement compositions of these embodiments may be further characterized in that the mode (as determined using the Horiba LA-300 laser diffraction particle sizer (Version 3.30 software for Windows 95) (Irvine, Calif.)) is 8.0 or less, such as 6.0 or less, e.g., 5 or less, including 3.0 μm or less. In some instances, the reactive α-tricalcium phosphate is produced by jet milling, e.g., as described in United States Published Patent Application 20070189951, the disclosure of which is herein incorporated by reference.

### Multi-Size Pore Forming Calcium Sulfate Dihydrate Component

In addition to reactive a-tricalcium phosphate, the dry reactants further include a multi-size pore forming calcium sulfate dihydrate component. Calcium sulfate dihydrate compositions of interest are particulate compositions of calcium sulfate dihydrate, where the calcium sulfate dihydrate may be present in the alpha and/or beta form. As summarized above, the calcium sulfate dihydrate composition is a multi-size pore forming calcium sulfate dihydrate component. The total amount of calcium sulfate dihydrate component in the dry reactants may vary, ranging in some instances from 5 to 50 wt. %, such as 15 to 35 wt. %, including 20 to 30 wt. %. By multi-size pore forming calcium sulfate dihydrate is meant that particulate calcium sulfate dihydrate composition includes particles that, upon production of the setting composition, provide for the production of at least two distinct pore size ranges, such as three distinct pore size ranges, including more than 3 distinct pore size ranges. In some instances, this component is selected to provide for a first pore size having an average diameter ranging from 1 to 100 μm, such as 5 to 50 μm, e.g., 5 to 35 μm; a second pore size having an average diameter ranging from 100 to 200 μm, and a third pore size having an average diameter ranging from 200 to 1000 μm, such as 200 to 500 μm, including 200 to 400 μm. In some instances, the particulate calcium sulfate dihydrate composition may be made up of a first population of particles having an average diameter ranging from 1 to 100 μm, such as 5 to 50 μm, e.g., 5 to 35 μm; a second population of particles having an average diameter ranging from 100 to 200 μm, and a third second population of particles having an average diameter ranging from 200 to 1000 μm, such as 200 to 500 μm, including 200 to 400 μm. Where the calcium sulfate dihydrate component includes three different populations, the amount of the first population may range from 10 to 50 wt. %, such as 15 to 45 wt. %; the amount of the second population may range from 10 to 50 wt. %, such as 15 to 45 wt. % and the amount of the third population may range from 10 to 50 wt. %, such as 15 to 45 wt. %.

### Demineralized Bone Matrix (DBM) Component

In addition to reactive a-tricalcium phosphate and multi-size pore forming calcium sulfate dihydrate components, the dry reactants further include a demineralized bone matrix (DBM) component. DBM components may be employed in any convenient format, such as but not limited to: a lyophilized form, etc. Any convenient DBM may be employed. The term DBM is employed to refer to any collagenous material entraining growth factors that natively occur in bone, such as one or more bone morphogenic proteins, e.g., BMP-2 and/or BMP-4, transforming growth factor-beta-1 (TGF-beta1), insulin-like growth factor-1 (IGF-1), or any combination of some or all of these. In this regard, as used herein, the term “demineralized bone matrix” includes a matrix material prepared by demineralizing any bone source, including cortical and/or cancellous bone. In some instances, DBM materials contain 5% or less by weight of residual calcium. The source bone can be from any suitable source including autogenic, allogenic, and/or xenogenic bone. When used in describing a DBM material, the term “osteoinductive” refers to the ability of the DBM material to induce bone growth. Alternatively, DBM materials can be provided lacking osteoinductive character, and nonetheless can be used as osteoconductive materials that provide a scaffold capable of receiving bone growth induced by natural healing processes or other materials implanted in the patient.

DBM materials for use in the present invention can be obtained commercially or can be prepared by any convenient protocol, several of which are well-known to those of skill in the art. In general, advantageous, osteoinductive DBM materials can be prepared by decalcification of cortical and/or cancellous bone, often by acid extraction. This process can be conducted so as to leave collagen, noncollagenous proteins, and growth factors together in a solid matrix. Methods for preparing such bioactive demineralized bone matrix are well known, in respect of which reference can be made to U.S. Pat. Nos. 5,073,373; 5,484,601; and 5,284,655, as examples. DBM products are also available commercially, including for instance, from sources such as Regeneration Technologies, Inc. (Alachua, Fla.), The American Red Cross (Arlington, Va.), and others. DBM materials that are solely osteoconductive can be prepared using similar techniques that have been modified or supplemented to remove or inactivate (e.g. by crosslinking or otherwise denaturing) components in the bone matrix responsible for osteoinductivity. Osteoinductive and/or osteoconductive DBM materials used in the present invention can desirably be derived from human donor tissue, especially in regard to implant devices intended for use in human subjects. It will be understood, however, that DBM materials can also be derived from non-human animal sources and used in implants intended for use in humans or other animals.

In certain embodiments, the particulate DBM material can have an average particle size of 1,000 μm or less. For instance, the DBM material can have particle sizes in the range of 50 to 850 μm, such as 125 to 850 μm, where in some instances the particle size range has an upper limit of 800 μm or less, such as 600 μm or less, e.g., 500 μm or less. In additional embodiments, the particulate DBM material can be in the form of elongate particles, such as fibers or ribbons. DBM ribbons having a median width of greater than about 0.5 mm are preferred, in certain embodiments with median lengths in the range of about 5 mm to about 20 mm and median thicknesses in the range of about 0.02 to about 0.2 mm. Illustratively, the DBM ribbon compositions can have median widths from about 0.5 mm to about 3 mm, median lengths of about 5 mm to about 20 mm, and median thicknesses of about 0.02 to about 0.2 mm. Such ribbon-form DBM particles can be made, for example, by milling off ribbons of bone from donor (e.g. human allograft) tissue, and then demineralizing the bone ribbons. Such milling can be conducted with a side-cutting bit. Alternatively or in addition, such DBM ribbons can be made by milling or grating the ribbons from a piece of demineralized cortical bone. Where ribbon-form DBM particles as described herein are used, effective formulations can be prepared which contain lower amounts of the collagen particles, or which are even free from the collagen particles. The elongate ribbon form of the DBM particles promotes entanglement which, along with the thickening and binding nature of the polysaccharide-containing liquid carrier, can be used to provide paste or putty compositions of good consistency and cohesiveness, and in particular putty compositions with beneficial resistance to deformation.

In certain embodiments, the particulate DBM incorporated into the inventive composition can include a substantial component of relatively larger DBM particles in combination with relatively smaller DBM particles. In certain aspects, the particulate DBM can be constituted at least 10 weight % by particles having a maximum dimension of greater than about 2 mm, or greater than about 3 mm (e.g. in the range of about 3 mm to about 5 mm) and at least 10 weight % by particles having a maximum dimension of less than about 1 mm. In further aspects, the particulate DBM can be constituted at least 20 weight % by particles having a maximum dimension of greater than about 2 mm, or greater than about 3 mm (e.g. in the range of about 3 mm to about 5 mm) and at least 20 weight % by particles having a maximum dimension of less than about 1 mm. In still further embodiments, the particulate DBM can be constituted about 10 weight % to about 40 weight % by particles having a maximum dimension of greater than about 2 mm, or greater than about 3 mm (e.g. in the range of about 3 mm to about 5 mm) and about 90 weight % to about 60 weight % by particles having a maximum dimension of less than about 1 mm. It will be understood that particles as described above may have the given dimensions along one axis or along two or three axes. Relatively volumetric particles can be used, for instance having shapes ranging from generally round to generally cuboidal. Products having such DBM particle size and/or shape distributions can be prepared, for example, by blending separate DBM products having the respective particle size distributions. The presence of relatively large DBM particles in combination with smaller particles can provide an overall composition that resists compression upon impingement by soft tissues at an implant site, and that also can exhibit beneficial handling and osteoinductive properties.

Insoluble collagen material for use in the invention can be derived from natural tissue sources (e.g. xenogenic, allogenic, or autogenic relative to the recipient human or other patient) or recombinantly prepared (e.g. recombinant human collagen). Collagens can be subclassified into several different types depending upon their amino acid sequence, carbohydrate content and the presence or absence of disulfide crosslinks. Types I and III collagen are two of the most common subtypes of collagen. Type I collagen is present in skin, tendon and bone, whereas Type III collagen is found primarily in skin. The collagen used in compositions of the invention can be obtained from skin, bone, tendon, or cartilage and purified by methods well known in the art and industry. Sources other than bone are preferred, in certain embodiments, for the collagen component of compositions of the invention. Alternatively, the collagen can be purchased from commercial sources. Type I bovine collagen is preferred for use in the invention.

The collagen can be a telopeptide collagen, and can be essentially free from protein materials other than collagen. Still further, either or both of non-fibrillar and fibrillar collagen can be used. Non-fibrillar collagen is collagen that has been solubilized and has not been reconstituted into its native fibrillar form. Suitable collagen products are available commercially, including for example from Kensey Nash Corporation (Exton, Pa.), which manufactures a fibrous collagen known as Semed F, from bovine hides. Collagen materials derived from bovine hide are also manufactured by Integra Life Science Holding Corporation (Plainsboro, N.J.). Naturally-derived or recombinant human collagen materials are also suitable for use in the invention. Illustratively, recombinant human collagen products are available from Fibrogen, Inc. (San Francisco, Calif.).

The solid particulate collagen incorporated into the inventive compositions can be in the form of intact or reconstituted fibers, or randomly-shaped particles, for example. In certain beneficial embodiments, the solid particulate collagen will be in the form of particles derived from a sponge material, for example by randomly fragmenting the sponge material by milling, shredding or other similar operations. Such particulated sponge material can have an average maximum particle diameter of less than about 6 mm, more preferably less than about 3 mm, and advantageously in the range of about 0.5 mm to 2 mm. Such materials can, for example, be obtained by milling or grinding a porous sponge material and sieving the milled or ground material through a screen having openings sized about 6 mm or smaller, desirably about 0.5 mm to about 2 mm. Retch grinders with associated sieves are suitable for these purposes. The resulting small sponge particles are randomly formed and have generally irregular shapes with remnant structures from the sponge material, and are highly beneficial for use in malleable compositions such as pastes or putties of the invention. In this regard, the use of such particulated sponge materials in combination with DBM materials in malleable compositions is considered as an inventive aspect disclosed herein also wherein the sponge material is made all or in part from a bioresorbable material other than collagen. For example, the particulated sponge material can be made from any of the other natural or synthetic polymers disclosed herein. Likewise, in these particulated sponge embodiments, the liquid carrier can be a polysaccharide-containing substance as disclosed herein or another suitable material, including aqueous and non-aqueous liquid mediums, and the particulated sponge material can optionally be used in the same relative amounts disclosed herein for the collagen solids materials. Further, a sponge starting material has been chemically crosslinked with an aldehyde crosslinker such as formaldehyde or glutaraldehyde, or another suitable chemical crosslinker such as a carbodiimide, or by other techniques such as dehydrothermal or radiation-induced crosslinking, the particulated collagen or other bioresorbable material retains the chemical crosslinking and provides an advantageous, lasting scaffold for bone ingrowth. Other sources of chemically crosslinked, particulate collagen, in fiber, irregular or other shapes, can also be used to significant advantage, and their use is considered to be another aspect of the present invention. These crosslinked particulate materials can be provided as starting materials for preparing compositions as disclosed herein, and therefore as incorporated in the device these particles are individually crosslinked. As well, crosslinked solid collagen particles can be used in combination with non-crosslinked collagen in compositions of the invention, wherein the non-crosslinked collagen can be solid (insoluble) or soluble collagen, or combinations thereof. Such crosslinked and non-crosslinked collagen mixtures can be used, for example, to modulate the residence time of the collagen portion of the implant compositions in vivo.

In other advantageous embodiments, the particulate collagen, crosslinked and/or non-crosslinked, can be in the form of elongate particles, such as fibers or ribbons. Collagen ribbons having a median width of greater than about 0.2 mm are preferred, more preferably greater than about 0.5 mm, in certain embodiments with median lengths in the range of about 5 mm to about 20 mm and/or median thicknesses in the range of about 0.02 mm to about 0.2 mm. Illustratively, the collagen ribbons can have median widths from about 0.2 mm to about 3 mm (more preferably 0.5 mm to about 3 mm), median lengths of about 5 mm to about 20 mm, and median thicknesses of about 0.02 mm to about 0.2 mm. When such elongate collagen ribbon compositions are used, potentially in conjunction with similarly-sized DBM ribbon compositions, or other DBM compositions described herein or otherwise, an advantageous mechanical entanglement of materials in the formulation can be achieved.

The amount of DBM component in the dry reactants may vary. In some instances, the DBM component may be present in an amount ranging from 5 to 25 wt. %, such as 5 to 20 wt. %, including 10 to 15 wt. % of the dry reactants.

### Calcium to Phosphate Ratio of Dry Reactant

The ratios or relative amounts of each of the disparate calcium and/or phosphate compounds (e.g., the reactive a-tricalcium phosphate and calcium sulfate dihydrate) in the dry reactant mixture is one that provides for the desired calcium phosphate product upon combination with the setting fluid and subsequent setting. In some embodiments, the overall ratio (i.e., of all of the disparate calcium and/or phosphate compounds in the dry reactants) of calcium to phosphate in the dry reactants ranges from 4:1 to 0.5:1, such as from 2:1 to 1:1 and including from 1.9:1 to 1.33:1.

### Setting Fluid Component

Another component of the cements is a setting fluid component. Setting fluids of interest include a variety of physiologically compatible fluids, including, but not limited to: water (including purified forms thereof, deionized forms thereof, etc.), aqueous alkanol solutions, e.g. glycerol, where the alkanol is present in minor amounts, e.g., 20 volume percent or less; pH buffered or non-buffered solutions; solutions of an alkali metal hydroxide, acetate, phosphate or carbonate, particularly sodium, more particularly sodium phosphate or carbonate, e.g., at a concentration in the range of 0.01 to 2M, such as from 0.05 to 0.5M, and at a pH in the range of 6 to 11, such as from 7 to 9, including from 7 to 7.5; and the like.

In certain embodiments, a silicate setting fluid, i.e., a setting fluid that is a solution of a soluble silicate, is employed. By solution of a soluble silicate is meant an aqueous solution in which a silicate compound is dissolved and/or suspended. The silicate compound may be any compound that is physiologically compatible and is soluble in water. By soluble in water is meant a concentration of 1% or more, such as 2% or more and including 5% or more, where the concentration of the silicate employed may range from 0-0.1 to 20%, such as from 0.01-5 to 15% and including from 5 to 10%. Silicate setting fluids finding use with calcium phosphate cements are further described in U.S. Pat. No. 6,375,935; the disclosure of which is herein incorporated by reference. Of interest in some instances is a sodium silicate setting solution. Sodium silicate setting solutions of interest include silica and sodium oxide. The concentration of silica may vary, ranging in some instances from 80 to 120 mM, such as 90 to 100 mM. The concentration of sodium oxide may vary, ranging in some instances from 15 to 50 mM, such as 20 to 30 mM. In some instances, the setting fluid is an alkali setting fluid, where the pH of the setting fluid in some instances is 8 or higher, such as 9 or higher, e.g., 10 or higher, including 11 or higher.

In some embodiments, the setting fluid includes a cellulose component, such that the setting fluid is a cellulosic setting fluid. Of interest are water-soluble cellulose components, where specific cellulose components of interest include, but are not limited to: nonionic cellulose ethers, such as but not limited to: methylcellulose, ethylcellulose, hydroxymethylcellulose, hydroxyethylcellulose, carboxymethylcellulose, carboxyethylcellulose and hydroxypropylcellulose; additional celluloses, such as carboxymethylcellulose sodium, carboxymethylcellulose calcium, etc. In certain embodiments, the cellulose is carboxymethylcellulose. Carboxymethylcellulose is available from a variety of commercial sources, including but limited to, Sigma, Hercules, Fluka and Noviant. In certain embodiments, the average molecular weight of the cellulose is 1000 daltons or higher, such as 5000 daltons or higher, where the average molecular weight may be as high as 10,000 daltons or higher, e.g., 50,000 daltons or higher, 100,000 daltons or higher, and ranges in certain embodiments from 5,000 to 100,000 daltons, such as from 10,000 to 50,000 daltons. While the concentration of the cellulose in the setting fluid may vary, in some instances the concentration ranges from 0.5 to 5, such as1 to 3 and including 2 to 3. In these instances, the setting fluid may be a fluid as described in U.S. patent application Ser. No. 12/771,999; the disclosure of which is herein incorporated by reference.

In some instances, the setting fluid is not a silicate setting fluid, i.e., the setting fluid does not include a silicate. As such, the setting fluid is not a silicate setting fluid as described in U.S. Pat. No. 6,375,935.

In certain embodiments, the setting fluid may further include an amount of phosphate ion, as described in U.S. Application Publication No. 20040250730; the disclosure of which is herein incorporated by reference in its entirety. For example, the concentration of phosphate ion in the setting fluid may vary, but may be 0.01 mol/L or greater, such 0.02 mol/L or greater and including 0.025 mol/L or greater, where the concentration may range from 0.01 to 0.5, such as from 0.01 to 0.25, including from 0.02 to 0.2 mol/L. The desired phosphate concentration may be provided using any convenient phosphate source, such as a non-calcium-containing salt of phosphoric acid that is sufficiently soluble, e.g., Na₃PO₄, Na₂HPO₄, or NaH₂PO₄. Salts of other cations such as K⁺, NH₄⁺, etc., may also be employed.

### Additional Optional Cement Components

One or both of the above liquid and dry reactant components may include an active agent that modulates the properties of the product into which the flowable composition prepared by the subject method sets. Such additional ingredients or agents include, but are not limited to: organic polymers, e.g., proteins, including bone associated proteins which impart a number of properties, such as enhancing resorption, angiogenesis, cell entry and proliferation, mineralization, bone formation, growth of osteoclasts and/or osteoblasts, and the like, where specific proteins of interest include, but are not limited to: osteonectin, bone sialoproteins (Bsp), α-2HS-glycoproteins, bone Gla-protein (Bgp), matrix Gla-protein, bone phosphoglycoprotein, bone phosphoprotein, bone proteoglycan, protolipids, bone morphogenic protein, cartilage induction factor, platelet derived growth factor, skeletal growth factor, and the like; particulate extenders; inorganic water soluble salts, e.g., NaCl, calcium sulfate; sugars, e.g., sucrose, fructose and glucose; pharmaceutically active agents, e.g., antibiotics; and the like. Of particular interest in certain embodiments are formulations that include the presence of one or more osteoinductive agents, including, but not limited to, those listed above. Additional active agents of interest include osteoclast induction agents, e.g., RANKL, as described in U.S. Pat. No. 7,252,833, the disclosure of which is herein incorporated by reference.

In some instances, an angiogenic factor is combined with the dry reactants and setting fluid, so that the flowable composition includes an amount of an angiogenic growth factor. As used herein, an “angiogenic growth factor polypeptide” refers to any protein, polypeptide, mutein or portion that is capable of inducing endothelial cell growth.

Angiogenic growth factors of interest include, but are not limited to: vascular endothelial cell growth factors (VEGF), acidic fibroblast growth factor (aFGF), basic fibroblast growth factor (bFGF), FGF2, epidermal growth factor, transforming growth factors α and β, platelet-derived endothelial growth factor, platelet-derived growth factor, tumor necrosis factor α, hepatocyte growth factor (scatter factor), erythropoietin, colony stimulating factor (CSF), macrophage-CSF (M-CSF), granulocyte/macrophage CSF (GM-CSF), angiopoietin 1 and 2, and nitric oxide synthase (NOS). The nucleic acid and amino acid sequences for these and other angiogenic growth factors are available in public databases such as GenBank and in the literature.

In some instances, the angiogenic growth factor is a VEGF, where VEGF proteins of interest include, but are not limited to: VEGF 1 (also referred to as VEGF A); VEGF 2 (also referred to as VEGF C); VEGF B; and VEGF D), PGF, etc. In addition to the above angiogenic growth factors, also of interest are their homologs and alleles and functionally equivalent fragments or variants thereof. For example, human VEGF 1 (VEGF A) exists in four principal isoforms, phVEGF₁₂₁; phVEGF₁₄₅; phVEGF₁₆₅; and phVEGF₁₈₉. Also of interest are the VEGF proteins and mutants thereof described in U.S. Pat. Nos. 5,851,989; 5,972,338; 057,428; 6,258,560; 6,348,351; 6,350,450; 6,368,853; 6,391,311; 6,395,707; 6,451,764; 6,455,496; 6,492,331; 6,551,822; 6,576,608; 6,586,397; 6,620,784; 6,750,044; 6,897,294; 6,927,024; 7,005,505; 7,060,278; 7,090,834; 7,208,472; 7,323,553; 7,427,596; 7,446,168; 7,494,977; 7,632,810; 7,651,703; 7,700,571; 7,709,455; 7,727,536; 7,785,588.

In some instances, the angiogenic factor (when present) may be complexed with an agent that modulates the release of the angiogenic factor from is the settable composition following implantation, i.e., a release modulatory agent. By “complexed with” is meant that the angiogenic factor and the release modulatory agent are intimately associated with each other. The nature of the intimate association of the angiogenic factor and the release modulatory agent may vary, where examples of intimate association include, but are not limited to: co-precipitation, encapsulation, dispersion, and the like, and may be achieved using a variety of different protocols, including but not limited to: co-precipitation, dip-coating, spray coating, solvent evaporation (lyophilization), etc.

The release modulatory agent may be any of a variety of different materials, so long as the materials are biocompatible and provide for the desired release modulatory activity. Release modulatory agent materials of interest include both inorganic and organic materials. Inorganic materials of interest include, but are not limited to: calcium phosphates, such as amorphous calcium phosphate crystalline hydroxyapatite. Organic materials of interest include, but are not limited to, organic polymers, e.g., alginates, chitosan, celluloses, PVA, PEG, gelatin, collagen, etc. Of interest are organic polymers that readily form gels and are cross-linkable at room or body temperature by common biocompatible methods. Where desired, the angiogenic factor/release modulatory agent complex may be further processed into a desirable composition format, for example a three dimensional structural configuration. Examples of three dimensional structural configurations of interest include, but are not limited to: gel micro-beads, fibers, foams, and the like.

In some instances, the dry reactant and/or setting fluid components further include a monovalent cation dihydrogen phosphate salt. By monovalent cation dihydrogen phosphate salt is meant a salt of a dihydrogen phosphate anion and a monovalent cation, e.g., K⁺, Na⁺, etc., where the salt may or may not include one or more water molecules of hydration, e.g., may be anhydrous, a monohydrate, a dihydrate, etc. The monovalent cation dihydrogen phosphate salts present in the cements of these embodiments of the invention may be described by the following formula:

Y⁺H₂PO₄.(H₂O)ₙ

where:

Y⁺ is a monovalent cation, such as K⁺, Na⁺, etc.; and

n is an integer from 0 to 2.

In certain embodiments, the salt is a sodium dihydrogen phosphate salt, such as sodium biphosphate (i.e., sodium phosphate monobasic, NaH₂PO₄), or the monohydrate (NaH₂PO₄.H₂O) or dihydrate (NaH₂PO₄.2H₂O) thereof.

The amount of monovalent cation dihydrogen phosphate salt that is present in the dry reactants may vary, but is in some instances present in an amount sufficient to provide for a rapidly setting high strength attainment composition, as described in greater detail below. In certain embodiments, the salt is present in an amount that ranges from 0.10 to 10 wt. %, such as from 0.2 to 5.0 wt. %, including from 0.5 to 5.0 wt. % of the total weight of the dry reactants. Further details regarding these salts and cements of interest that include the same are provided in United States Published Patent Application No. 20050260279, the disclosure of which is herein incorporated by reference.

Where desired, a cyclodextrin may be present in the composition prepared from the dry reactants and the setting fluid. Depending on the desired format, the cyclodextrin may be present in the dry reactants or in the setting fluid. By cyclodextrin is meant a cyclic oligosaccharide or mixture of cyclic oligosaccharides, composed of 5 or more α-D-glucopyranoside units that exhibit a 1->4 linkage. Cyclodextrins of interest include α-cyclodextrin, β-cyclodextrin and γ-cyclodextrin. The amount of cyclodextrin that is present in either the liquid or dry components may vary, depending on the amount that is desired in the flowable composition produced therefrom. In some instances, the amount of cyclodextrin that is desired in the flowable composition produced upon combination of the dry reactants and setting fluid ranges from 0.01 to 10% (w/w), such as 0.05 to 2.0% (w/w). In some instances where the cyclodextrin is present in the dry reactant component, the amount of cyclodextrin that is present in the dry reactant component ranges from 0.01 to 10% by weight, such as 0.05 to 2.0% by weight. Cyclodextrin components and details regarding the same are further described in U.S. patent application Ser. No. 12/568,531; the disclosure of which is herein incorporated by reference.

In certain embodiments, the cement may further include a contrast or imaging agent, where the contrast agent may be present in one or both of the liquid and dry components, or separate therefrom until combination of all of the components to produce the flowable composition. Contrast agents of interest include, but are not limited to: the water soluble contrast agents described in U.S. Pat. No. 7,306,786, the disclosure of which is herein incorporated by reference in its entirety; and the barium apatite contrast agents described in U.S. application Ser. No. 10/851,766 (Published as US20050257714), the disclosure of which is herein incorporated by reference in its entirety.

In certain embodiments, the subject cement compositions may be seeded with any of a variety of cells, as described in published U.S. Patent Publication No. 20020098245, the disclosure of which is herein incorporated by reference in its entirety.

In certain embodiments, the dry reactants are further characterized by including a second reactant (a coarse particle reactant) that has a mean particle size that is 2 times or more larger than the mean particle size of the first reactant component, where the mean particle size of this second reactant may be 9 μm or larger, such as 10 μm or larger, including 20 μm or larger, e.g., 25 μm or larger, 30 μm or larger (as determined using the Horiba LA-300 laser diffraction particle sizer (Version 3.30 software for Windows 95) (Irvine, Calif.)) such as 50 μm or larger, 100 μm or larger, 150 μm or larger, 200 μm or larger, where the particle size of the tricalcium phosphate coarse particle component population (also referred to herein as a coarse particle size population) may range from 10 to 500 μm, such as from 25 to 250 μm. In certain instances, the particles of this component can range in size from 38 μm to 212 μm, such as from 38 μm to 106 μm or 106 μm to 212 μm. In some instances, this coarse particle component is manufactured using the protocol described in U.S. Published Patent Application No. 2010-0143480; the disclosure of which is herein incorporated by reference.

In certain embodiments, the amount of the first reactant component of the dry reactant composition is greater than the total amount of other reactant components that may be present, such as the second reactant component as described above. In certain of these embodiments, the mass ratio of the first reactant component to the total mass of the dry reactants may range from 1 to 10, e.g., from 9 to 6, such as from 9 to 7, including from 9.5 to 8.5.

In certain embodiments, the dry reactants may further include an amount of an emulsifying agent, as described in U.S. application Ser. No. 11/134,051 (published as US 2005-0260279); the disclosure of which is herein incorporated by reference in its entirety. Emulsifying agents of interest include, but are not limited to: polyoxyethylene or polyoxypropylene polymers or copolymers thereof, such as polyethylene glycol and polypropylene glycol; nonionic cellulose ethers such as methylcellulose, ethylcellulose, hydroxymethylcellulose, hydroxyethylcellulose, carboxymethylcellulose, carboxyethylcellulose and hydroxypropylcellulose; additional celluloses, such as carboxymethylcellulose sodium, carboxymethylcellulose calcium, carboxymethylstarch; polysaccharides produced by microbial fermentation, such as yeast glucans, xanthan gum, β-1,3-glucans (which may be straight-chained or branched; e.g. curdlan, paramylum, pachyman, scleroglucan, laminaran); other natural polymers, e.g., gum arabic, guar gum, carrageenin, gum tragacanth, pectin, starch, gelatin, casein, dextrin, cellulose; polyacrylamide; polyvinyl alcohol; starch; starch phosphate; sodium alginate and propylene glycol alginate; gelatin; amino-containing acrylic acid copolymers and quaternization products derived therefrom; and the like.

In certain embodiments, the emulsifying agent is a cellulose ether, particularly a nonionic cellulose ether, such as carboxymethylcellulose. Carboxymethylcellulose is available from a variety of commercial sources, including but limited to, Sigma, Hercules, Fluka and Noviant. In certain embodiments, the average molecular weight of the cellulose ether is 1000 daltons or higher, such as 5000 daltons or higher, where the average molecular weight may be as high as 10,000 daltons or higher, e.g., 50,000 daltons or higher, 100,000 daltons or higher, and ranges in certain embodiments from 5,000 to 100,000 daltons, such as from 10,000 to 50,000 daltons. When present, the proportion of the emulsifying agent in the dry reactant in certain embodiments ranges from 0.01 to 10% (w/w), such as from 0.05 to 2.0% (w/w), e.g., 0.1 to 1% (w/w).

### Methods of Combining Cement Components to Produce a Settable Composition

In producing settable compositions of the invention, e.g., compositions that are suitable for implantation, suitable amounts of the dry reactant and the setting fluid components are combined to produce the settable composition, where the settable composition sets into a solid product following implantation. The ratio of the dry reactants to setting fluid (i.e. the liquid to solids ratio) is selected to provide for an initial “flowable” composition that is also sellable, where by “sellable” is meant that the composition goes from a first non-solid (and also non-gaseous) state (i.e., flowable state) to a second, solid state after setting. In certain embodiments, the liquid to solids ratio is chosen to provide for a flowable composition that has a viscosity ranging from that of bovine whole milk to that of modeling clay. As such, the liquids to solids ratio employed in the subject methods ranges in some instances from 0.2 to 1.0, such as from 0.3 to 0.6. Of interest in certain embodiments are methods that produce a paste composition, where the liquid to solids ratio employed in such methods may range from 0.25 to 0.5, such as from 0.3 to 0.45.

Mixing may be accomplished using any convenient protocol, including manual mixing (e.g., as described in U.S. Pat. No. 6,005,162 and automated mixing (e.g., as described in WO 98/28068), the disclosures of which publications are herein incorporated by reference. Also of interest is vibratory mixing, e.g., as described in U.S. Pat. Nos. 7,261,717; 7,252,672 and 7,261,718, the disclosures of which are herein incorporated by reference.

The temperature of the environment in which combination or mixing of the dry and liquid components takes place is sufficient to provide for a product that has desired setting and strength characteristics, and may range from 0 to 50° C., such as from 15 to 30° C., including 15 to 25° C., e.g., 16 to 18.5° C. or 22.5 to 25° C. In certain instances, mixing occurs at a temperature that is: 15° C., 16° C., 17° C., 18° C., 19° C., 20° C., 21° C., 22° C., 23° C., 24° C. and 25° C., or a temperature in between any sequential two of these temperatures.

Mixing takes place for a period of time sufficient for a flowable composition to be produced, and may take place for a period of time ranging from 15 to 120 seconds, such as from 15 to 100 seconds and including from 15 to 60 seconds, e.g., 15 to 50 seconds, 15 to 30 seconds, etc.

### Settable Composition

The above-described protocols result in the production of a sellable composition that is capable of setting into a calcium phosphate mineral product, e.g., as described in greater detail below. The settable compositions produced by the above-described methods are compositions that set into a biologically compatible, and often resorbable and/or remodelable, product, where the product is characterized by including calcium phosphate molecules not present in the initial reactants, i.e., that are the product of a chemical reaction among the initial reactants.

Prior to setting, the settable compositions are flowable. The term “flowable” is meant to include paste-like compositions, as well as more liquid compositions (e.g., compositions having a lower viscosity). As such, the injectable viscosity time period of the subject flowable compositions, defined as the time period during which the mixed composition can be injected through a standard Luer-lok fitting after mixing, may range from up to 10 minutes, such as up to 9 minutes, such as up to 8 minutes, such as up to 7 minutes, such as up to 6 minutes, such as up to 5 minutes, and including up to 4 minutes. Of interest in certain embodiments are paste compositions that have an injectable viscosity time period ranging from up to 5 minutes, such as up to 4 minutes. Pastes that stay paste-like for longer periods may be displaced by bleeding bone once implanted into the body, which create a blood interface between the cement and the bone prior to the cement hardening.

The compositions produced according to embodiments of the invention set into calcium phosphate mineral containing products. By “calcium phosphate mineral containing” product is meant a solid product that includes one or more, usually primarily one, calcium phosphate mineral. In certain embodiments, the calcium phosphate mineral is one that is generally poorly crystalline, so as to be resorbable and, often, remodelable, over time when implanted into a physiological site. The calcium to phosphate ratio in the product may vary depending on particular reactants and amounts thereof employed to produce it, and in some instances ranges from 2:1 to 1.33:1, such as from 1.8:1 to 1.5:1 and including from 1:7:1 to 1.6:1. Of interest in certain embodiments are apatitic products, which apatitic products have a calcium to phosphate ratio ranging from 2.0:1 to 1.33:1, including both hydroxyapatite and calcium deficient analogs thereof, including carbonate substituted hydroxyapatite (i.e. dahllite), etc.

The period of time required for the compositions to harden or “set” may vary. Set time is determined using the Gilmore Needle Test (ASTM C266-89), modified with the cement submerged under 37° C. physiological saline. The set times of the subject cements may range from 30 seconds to 30 minutes, such as from 2 to 15 minutes and including from 4 to 12 minutes. In certain embodiments, the settable composition sets in a clinically relevant period of time. By clinically relevant period of time is meant that the paste-like composition sets in less than 20 minutes, usually less than 15 minutes and often in less than 10 minutes, where the composition remains flowable for 1 minute or longer, usually 2 minutes or longer and, in many embodiments, for 5 minutes or longer following combination or mixture of the precursor liquid and dry cement components.

In some instances, the compositions rapidly set into a high strength product, as determined by the ASTM C403/C403M-06 modified test described in U.S. patent application Ser. No. 12/771,999; the disclosure of which is herein incorporated by reference. In some instances, the compositions attain high strength rapidly, such that they may be viewed as rapid strength attainment compositions. As such, at 6 minutes the compositions of certain embodiments have a setting value of 150 Newtons or greater, such as 300 Newtons or greater, where in some embodiments the setting strength at 6 minutes ranges from 150 to 500 Newtons. At 10 minutes the compositions may have a setting value of 200 Newtons or greater, such as 300 Newtons or greater, including 400 Newtons or greater. At 15 minutes the compositions may have a setting value of 450 Newtons or greater, such as 500 Newtons or greater, including 600 Newtons or greater

The compressive strength of the product into which the settable composition sets may vary significantly depending on the particular components employed to produce it. Of particular interest in many embodiments is a product that has a compressive strength sufficient for it to serve as at least a cancellous bone structural material. By cancellous bone structural material is meant a material that can be used as a cancellous bone substitute material as it is capable of withstanding the physiological compressive loads experienced by compressive bone under at least normal physiological conditions. As such, the subject flowable paste-like material is one that sets into a product having a compressive strength of 1.5 MPa or greater, e.g., 2 MPa or greater, including 3 MPa or greater, e.g., 5 MPa or greater, as measured by the assay described in Morgan, E F et al., 1997, Mechanical Properties of Carbonated Apatite Bone Mineral Substitute Strength, Fracture and Fatigue Behavior. J. Materials Science: Materials in Medicine. V. 8, pp 559-570.

The resultant product may have a high tensile strength. Tensile strength is determined using the protocol described in U.S. patent application Ser. No. 12/771,999 (the disclosure of which is herein incorporated by reference), and where the products may exhibit a 24-hour tensile strength of 0.5 MPa or greater, e.g., 1 MPa or greater, including 2.5 MPa or greater, e.g., 5 MPa or greater, such as 6 MPa or greater, e.g., 7.5 to 8 MPa, where in some instances the tensile strength ranges from 0.5 to 6.0 MPa.

In certain embodiments, the resultant product is stable in vivo for extended periods of time, by which is meant that it does not dissolve or degrade (exclusive of the remodeling activity of osteoclasts) under in vivo conditions, e.g., when implanted into a living being, for extended periods of time. In these embodiments, the resultant product may be stable for 4 months or longer, 6 months or longer, 1 year or longer, e.g., 2.5 years, 5 years, etc. In certain embodiments, the resultant product is stable in vitro when placed in an aqueous environment for extended periods of time, by which is meant that it does not dissolve or degrade in an aqueous environment, e.g., when immersed in water, for extended periods of time. In these embodiments, the resultant product may be stable for 4 months or longer, 6 months or longer, 1 year or longer, e.g., 2.5 years, 5 years, etc.

In certain embodiments, the flowable paste-like settable composition is capable of setting in a fluid environment, such as an in vivo environment at a bone repair site. As such, the flowable paste composition can set in a wet environment, e.g., one that is filled with blood and other physiological fluids. Therefore, the site to which the flowable composition is administered during use need not be maintained in a dry state.

Implanted compositions produced from the settable compositions as described above have a porosity profile that is determined by the multi-size pore forming calcium sulphate dihydrate component. The phrase “porosity profile” as used herein describes the nature of the porosity in the final product following setting, wherein in some instances the porosity profile may also refer to the time period over which the pores form, i.e., how long it takes for the pores to form following implantation (i.e., T₀).

The term “porosity” as used herein, refers to the average amount of non-solid space contained in a material (e.g., a composite of the present invention). Such space is considered void of volume even if it contains a substance that is liquid at ambient or physiological temperature, e.g., 0.5° C. to 50° C. Porosity or void volume of a composite can be defined as the ratio of the total volume of the pores (i.e., void volume) in the material to the overall volume of composites. In some instances, porosity (ε), defined as the volume fraction pores, can be calculated from composite foam density, which can be measured gravimetrically.

In some instances, the porosity profile of a set composition includes a collection of micropores and macropores present in the composition following a predetermined amount of time following implantation of the material. Micropores are pores having a diameter ranging from 0.1 to 1 μm, such as 0.1 to 0.5 μm. Macropores are pores having a diameter ranging from 1 to 1000 μm, such as 1 to 500 μm. As both micropores and macropores are present, the composition is both macroporous and microporous following a period of time after implantation. The ratio of micropores to macropores following a period of time after implantation may vary, ranging in some instances from 1:10 to 10:1. In some instances, the appearance of pores (micropores and/or macropores) in sufficient number to measurably impact (as measured by mercury porosimetry) the compressive and tensile strength of the implanted product does not occur for a period of time following implantation of 24 hrs or longer. In some instances, the set product includes pores having sizes that correspond to the sizes of the calcium sulfate dihydrate component employed to produce the product.

In some instances, the sellable compositions may be viewed as controlled pore forming calcium phosphate sellable compositions. By “controlled pore forming” is meant that the calcium phosphate sellable compositions assume a known porosity profile in a known amount of time following implantation and setting. In other words, the sellable compositions assume a predetermined porosity profile in a known amount of time in situ following introduction to a body site, i.e., T₀.

The total porosity of the set product, e.g., as determined by mercury porosimetry, may vary, and in some instances will range from 30 to 90%, such as 40 to 85%, where in some instances the porosity is 45% or greater.

### Applications

Settable compositions produced from cements of the invention, e.g., as described above, find use in applications where it is desired to introduce a flowable material capable of setting up into a solid calcium phosphate product into a physiological site of interest, such as in dental, craniomaxillofacial and orthopedic applications. In orthopedic applications, the cement may be prepared, as described herein, and introduced or applied to a bone repair site, such as a bone site comprising cancellous and/or cortical bone. In some instances, the site of application is a cancellous bone void that results from reducing a fracture. In these instances, the methods may include reducing a bone fracture and then applying an amount of the flowable composition to the resultant void, where the amount may be sufficient to substantially if not completely fill the void.

Orthopedic applications in which the cements prepared by the subject system find use include, but are not limited to, the treatment of fractures and/or implant augmentation, in mammalian hosts, particularly humans. In such fracture treatment methodologies, the fracture is first reduced. Following fracture reduction, a flowable structural material prepared by the subject system is introduced into the cancellous tissue in the fracture region using the delivery device described above. Specific dental, craniomaxillofacial and orthopedic indications in which the subject invention finds use include, but are not limited to, those described in U.S. Pat. Nos. 6,149,655; 6,375,935; 6,719,993; 7,175,858; 7,252,833; 7,252,841; 7,252,672; 7,261,717; 7,306,786; 7,658,940; 7,658,940; and U.S. patent application Ser. Nos. 12/328,720; 12/568,531; and 12/771,999; the disclosures of which patents and patent applications are herein incorporated by reference in their entirety. In yet other embodiments, the subject compositions find use in drug delivery, where they are capable of acting as long lasting drug depots following administration to a physiological site. See e.g. U.S. Pat. Nos. 5,904,718 and 5,968,253; the disclosures of which are herein incorporated by reference in their entirety.

### Kits

Also provided are kits that include the subject cements, where the kits at least include a dry reactant component and a setting fluid component, e.g., as described above. When both a dry component and setting fluid are present, the dry component and setting fluid may be present in separate containers in the kit, or some of the components may be combined into one container, such as a kit wherein the dry components are present in a first container and the liquid components are present in a second container, where the containers may or may not be present in a combined configuration, as described in U.S. Pat. No. 6,149,655, the disclosure of which is herein incorporated by reference. In addition to the cement compositions, the subject kits may further include a number of additional reagents, e.g., cells (as described above, where the composition is to be seeded), protein reagents (as described above), emulsifying agents, cyclodextrins, contrast agents, and the like.

In certain embodiments, the kits may further include mixing and/or delivery elements, e.g., mortar and pestle, spatula, etc., which elements find use in, e.g., the preparation and/or delivery of the cement composition.

In addition to above-mentioned components, the subject kits typically further include instructions for using the components of the kit to practice the subject methods. The instructional material may also be instructional material for using the cement compositions, e.g., it may provide surgical techniques and principals for a particular application in which the cement is to be employed. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, portable flash drive, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Systems

Also provided are systems that find use in practicing the subject methods, as described above. The subject systems at least include dry and liquid components of a cement, e.g., as described above, and a mixing element. In certain embodiments, the systems may further include additional agents, e.g., contrast agents, active agents, etc., as described above.

The following examples are offered by way of illustration and not by way of limitation.

### EXPERIMENTAL

### A. Formulation

1. Powder components:
α-tricalcium phosphate (TCP): 4 um
Calcium sulfate dihydrate powder (CSD): 38 um (with larger particle sizes, <500 um, as desired to provide for desired variation in pore size)
Sodium phosphate mono basic (SPMA): ground
Carboxymethyl Cellulose (CMC)
Demineralized Bone Matrix (DBM): 125-850 um (with potential for narrower particle range, e.g. <500 um)
   2. Current Wt. Percent:

Solid fraction:
56.1% TCP
28.0% CSD
4.8% SPMA
0.7% CMC
10.5% DBM

### 3. Liquid:

Sodium silicate soln. pH 11.1
   92 mM silica
   28 mM sodium oxide
      B. Cement preparation

The dry and liquid components were combined using a mortar and pestle to produce a paste composition.

### C. Results

1. Setting strength
a. Procedure

A modification of the standard setting test described in ASTM C403/C403M-06 is employed, in which the load required to drive needles a prescribed distance into concrete or a similar setting material is measured. The modification involves a needle with a tip configuration similar to that used in ASTM C266-07. A modified high load indentor (7 mm in diameter) is attached to Instron material testing machine with a maximum load of 5000 N. The needle is pushed 1.25 mm at a rate of 15.2 mm/s into the sample cured at 32±0.5° C. and 100% RH. No spring load average is calculated or used in later calculations (the high load indentor test fixture does not use a spring).

b. Results

Bone Void Filler + DBM

| | (N) |
|---|---|
| 6 minutes | 150 |
| 10 minutes | 250 |
| 15 minutes | 450 |

2. Tensile strength
a. Procedure:

The testing was conducted using an Instron mechanical testing system (Canton, Mass.). The test specimens were circular rings of 0.5″ I.D. and 0.3″ thickness that were filled with the cement using a spatula. The filled molds were placed into a phosphate buffered saline bath maintained at 37° C. and allowed to cure for 24 hours. Samples were then removed from the unit, placed on a steel platen and crushed at a cross head speed of 0.1 inches/minute. Ultimate tensile stress was calculated using the following equation:

σ=2P/πDt Equation of tensile stress:

where:
P=ultimate compressive load, Newtons
D=sample diameter, millimeters
t=sample thickness, millimeters.
b. Results

Using the above protocol, the tensile strength was observed to be 1.5 MPa

3. Compressive strength
a. Procedure

The compressive strength test is a modification of ASTM F 451. The primary difference from the ASTM method is that pressurization of the void filler specimens is not required. Additional modifications to the test involve curing the bone void filler specimens for 24 hours in a 37° C. phosphate buffered saline environment at pH=7.4 and sanding the ends of the specimens before removing them from the mold for testing. Each specimen is placed between the loading platens of the mechanical testing system. Specimens are loaded along the longitudinal axis at displacement rate of 0.1 in./min until failure. Load, displacement, and time are recorded continuously at a sampling rate of 10 Hz.

b. Results

Using the above protocol, the compressive strength was observed to be 6 MPa

All publications and patent applications mentioned in this specification are herein incorporated by reference to the same extent as if each individual publication or patent application was specifically and individually indicated to be incorporated by reference.

The invention now being fully described, it will be apparent to one of skill in the art that many changes and modifications can be made thereto without departing from the spirit and scope of the appended claims.

What is claimed is:

1. A method of producing a settable composition that sets into a calcium phosphate containing product, the method comprising:
combining:
(a) a dry reactant component comprising
(i) a reactive a-tricalcium phosphate component;
(ii) a multi-size pore forming calcium sulfate dihydrate component; and
(iii) a demineralized bone matrix (DBM) component; and
(b) a setting fluid component;
in a ratio of (a) to (b) sufficient to produce the sellable composition.

2. The method according to claim 1, wherein the reactive a-tricalcium phosphate component has a mean particle size of 8 μm or less.

3. The method according to claim 2, wherein the reactive a-tricalcium phosphate component is a jet-milled component.

4. The method according to claim 1, wherein the multi-size pore forming calcium sulfate dihydrate component comprises at least two distinct particle size ranges.

5. The method according to claim 4, wherein the multi-size pore forming calcium sulfate dihydrate component comprises at least three distinct particle size ranges.

6. The method according to claim 5, wherein the multi-size pore forming calcium sulfate dihydrate component comprises:
a first particle size ranging from 5 to 35 μm;
a second particle size ranging from 100-200 μm; and
a third particle size ranging from 200-400 μm.

7. The method according to claim 1, wherein the DBM component is a mammalian DBM component.

8. The method according to claim 7, wherein the mammalian DBM component is a human DBM component.

9. The method according to claim 1, wherein the setting fluid is water.

10. The method according to claim 1, wherein said sellable composition is a paste.

11. A kit comprising:
(a) a dry reactant component comprising
(i) a reactive a-tricalcium phosphate component;
(ii) a multi-size pore forming calcium sulfate dihydrate component; and
(iii) a demineralized bone matrix (DBM) component; and
(b) a setting fluid component;
in a ratio of (a) to (b) sufficient to produce the settable composition upon combination of the dry reactant and setting fluid components.

12. The kit according to claim 11, wherein the reactive a-tricalcium phosphate component has a mean particle size of 8 μm or less.

13. The kit according to claim 12, wherein the reactive a-tricalcium phosphate component is a jet-milled component.

14. The kit according to claim 1, wherein the multi-size pore forming calcium sulfate dihydrate component comprises at least two distinct particle size ranges.

15. The kit according to claim 14, wherein the multi-size pore forming calcium sulfate dihydrate component comprises at least three distinct particle size ranges.

16. The kit according to claim 15, wherein the multi-size pore forming calcium sulfate dihydrate component comprises:
a first particle size ranging from 5 to 35 μm;
a second particle size ranging from 100-200 μm; and
a third particle size ranging from 200-400 μm.

17. The kit according to claim 11, wherein the DBM component is a mammalian DBM component.

18. The kit according to claim 17, wherein the mammalian DBM component is a human DBM component.

19. A settable composition that sets into a calcium phosphate containing product, wherein said composition is prepared by combining:
(a) a dry reactant component comprising
(i) a reactive a-tricalcium phosphate component;
(ii) a multi-size pore forming calcium sulfate dihydrate component; and
(iii) a demineralized bone matrix (DBM) component; and
(b) a setting fluid component;
in a ratio of (a) to (b) sufficient to produce the settable composition.

20. A method of repairing a hard tissue defect, the method comprising applying to the site of the defect a flowable composition according to claim 19.
